(19) United States
(12) Patent Application Publication
(10) Pub. No.: US 2026/0315759 A1
(43) Pub. Date: Oct. 15, 2026

(54) METHOD OF TREATING AN OPHTHALMIC ARTERY STENOSIS

(71) Applicants: STRYKER CORPORATION, Portage, MI (US); STRYKER EUROPEAN OPERATIONS LIMITED, Carrigtwohill (IE)

(72) Inventors: Brent Charles Gerberding, San Jose, CA (US); Murtagh M. Murphy, Cork (IE)

(73) Assignees: STRYKER CORPORATION, Portage, MI (US); STRYKER EUROPEAN OPERATIONS LIMITED, Carrigtwohill (IE)

(21) Appl. No.: 19/693,279

(22) Filed: May 29, 2026

Related U.S. Application Data
(63) Continuation of application No. PCT/US2024/059045, filed on Dec. 6, 2024.
(60) Provisional application No. 63/609,817, filed on Dec. 13, 2023.

(51) Int. Cl. A61M 25/10; A61F 2/958; A61M 25/00; A61M 25/09

(52) U.S. Cl. CPC A61M 25/104; A61F 2/958; A61M 2025/0042; A61M 25/09; A61M 2210/12

(57) ABSTRACT

A method of treating an ophthalmic artery lesion, includes: advancing a distal end of a guidewire inside a patient into an ophthalmic artery through an ostium of the ophthalmic artery until the distal end of the guidewire passes a lesion in the ophthalmic artery; advancing a microcatheter over the guidewire until a distal end of the microcatheter is in the ophthalmic artery; removing the guidewire from within the microcatheter while the distal end of the microcatheter is in the ophthalmic artery; advancing a balloon catheter inside the microcatheter, wherein the balloon catheter comprises a balloon, and wherein the balloon catheter is without any guidewire lumen; deploying the balloon of the balloon catheter outside the microcatheter; and expanding the balloon to press against the lesion in the ophthalmic artery to create a compressed lesion.

### RELATED APPLICATION DATA

This application is a continuation of International Patent Application No. PCT/US2024/059045 filed on Dec. 6, 2024, pending, which claims priority to, and the benefit of U.S. Provisional Patent Application No. 63/609,817 filed on Dec. 13, 2023, lapsed. The entire disclosures of the above applications are expressly incorporated by reference herein.

### FIELD

The field of the disclosure relates to medical devices and methods, and more specifically, to a method of treating ophthalmic artery stenosis with a medical system designed to access small artery.

### BACKGROUND

Carotid artery stenosis is a narrowing of the carotid artery. The carotid artery delivers oxygenated blood from the heart to the brain. Narrowing of the carotid artery can cause stroke or other conditions.

Age-related macular degeneration (AMD) is an eye disease that can blur a person's central vision. It can happen when aging causes damage to the macula, which is the part of the eye that controls sharp, straight-ahead vision. Sometimes, a person's vision may be negatively affected by the narrowing of ophthalmic artery that extends from the internal carotid artery. When the ophthalmic artery is blocked, it can cause the patient to lose his/her eyesight. This adverse condition, known as central retinal artery occlusion, can happen suddenly without any pain. In some cases, AMD may be related to ophthalmic artery stenosis (lesion) and may be treated by cannulizing the ophthalmic artery.

Ophthalmic artery stenosis is more difficult to treat than carotid artery stenosis. This is because the access to the ophthalmic artery requires a challenging navigation from the internal carotid artery into the ophthalmic artery via the ostium (opening) of the ophthalmic artery. This navigation is particularly challenging because the size of the ophthalmic artery ostium is quite small when compared to the internal carotid artery, especially if the ophthalmic artery stenosis occurs at the ostium (opening) of the ophthalmic artery. Also, the ophthalmic artery extends from the internal carotid artery at an angle that makes navigation of a treatment device from the internal carotid artery into the ophthalmic artery difficult. In addition, the ophthalmic artery traverses adjacent to the optic nerve, thus increasing the risk of mechanical trauma to the artery and/or nerve when treating ophthalmic artery stenosis. The difficulty in treating ophthalmic artery stenosis due to the acute take-off angle of the ophthalmic artery, and due to the lack of intravascular catheter-based devices that are designed specifically to access the ophthalmic artery are well-documented in “Ophthalmic artery angioplasty for age-related macular degeneration” by Ivan Lylyk et al.

New systems and techniques for treating ophthalmic artery stenosis are described herein. The new systems and techniques allow treatment of ophthalmic artery stenosis to be performed easily, and minimize or at least reduce the risk of mechanical trauma to the artery and/or nerve in the treatment of ophthalmic artery stenosis.

### SUMMARY

A method of treating an ophthalmic artery lesion, includes: advancing a distal end of a guidewire inside a patient into an ophthalmic artery through an ostium of the ophthalmic artery until the distal end of the guidewire passes a lesion in the ophthalmic artery; advancing a microcatheter over the guidewire until a distal end of the microcatheter is in the ophthalmic artery; removing the guidewire from within the microcatheter while the distal end of the microcatheter is in the ophthalmic artery; advancing a balloon catheter inside the microcatheter, wherein the balloon catheter comprises a balloon, and wherein the balloon catheter is without any guidewire lumen; deploying the balloon of the balloon catheter outside the microcatheter; and expanding the balloon to press against the lesion in the ophthalmic artery to create a compressed lesion.

Optionally, the method further includes: deflating the balloon; and removing the balloon catheter from within the patient.

Optionally, the method further includes advancing the distal end of the guidewire inside the patient again into the ophthalmic artery through the ostium of the ophthalmic artery until the distal end of the guidewire passes the compressed lesion.

Optionally, the method further includes advancing the microcatheter over the guidewire again until the distal end of the microcatheter is in the ophthalmic artery.

Optionally, the method further includes: placing a stent-delivery device inside the microcatheter; and using the stent-delivery device to deploy a stent out of the distal end of the microcatheter, wherein the deployed stent compresses against the lesion.

Optionally, the method further includes advancing a stent catheter over the guidewire until a distal end of the stent catheter is in the ophthalmic artery.

Optionally, the method further includes: removing the guidewire from within the stent catheter while the distal end of the stent catheter is in the ophthalmic artery; and deploying a stent out of the distal end of the stent catheter, wherein the deployed stent compresses against the lesion.

Optionally, the method further includes deploying a stent out of the distal end of the stent catheter, wherein the deployed stent compresses against the lesion.

Optionally, the method further includes advancing a guide catheter inside the patient until a distal end of the guide catheter is in an internal carotid artery facing the ostium of the ophthalmic artery;

Optionally, the act of advancing the distal end of the guidewire is performed while a part of the guidewire is inside the guide catheter.

Optionally, the act of advancing the microcatheter over the guidewire is performed while at least a part of the microcatheter is inside the guide catheter.

Optionally, when the microcatheter is advanced, the distal end of the guide catheter remains in the internal carotid artery outside the ophthalmic artery.

Optionally, the act of advancing the balloon catheter inside the microcatheter is performed without placing the balloon catheter over the guidewire or any other guidewire.

Optionally, the act of deploying the balloon comprises retracting the microcatheter proximally or advancing the balloon catheter distally.

Optionally, the method is performed using a medical system that comprises the guidewire, the microcatheter, and the balloon catheter.

Optionally, the distal end of the microcatheter has a dimension that allows the distal end of the microcatheter to be placed inside the ophthalmic artery.

Optionally, the distal end of the guidewire is capable of accessing the ophthalmic artery from the internal carotid artery.

Optionally, the medical system also comprises a stent delivery device configured to deliver a stent into the ophthalmic artery.

A medical system for treating an ophthalmic artery lesion, includes: a guidewire having a distal end, wherein the distal end of the guidewire is configured to be advanced inside a patient into an ophthalmic artery through an ostium of the ophthalmic artery until the distal end of the guidewire passes a lesion in the ophthalmic artery; and a microcatheter configured to be advanced over the guidewire until a distal end of the microcatheter is in the ophthalmic artery; wherein the guidewire is removable from within the microcatheter while the distal end of the microcatheter is in the ophthalmic artery; wherein the medical system further comprises a balloon catheter configured to be advanced inside the microcatheter, wherein the balloon catheter comprises a balloon, wherein the balloon catheter is without any guidewire lumen; wherein the balloon of the balloon catheter is deployable outside the microcatheter; and wherein the balloon is expandable to press against the lesion to create a compressed lesion.

Optionally, the medical system further includes a stent delivery device configured to deliver a stent in the ophthalmic artery to press against the compressed lesion.

Other and further aspects and features will be evident from reading the following detailed description.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 illustrates a medical system configured to treat an ophthalmic artery stenosis.

FIGS. 2-9 illustrate a method of treating an ophthalmic artery lesion.

FIG. 10 is a block diagram showing a method of treating an ophthalmic artery lesion.

### DETAILED DESCRIPTION

Various embodiments are described hereinafter with reference to the figures. It should be noted that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

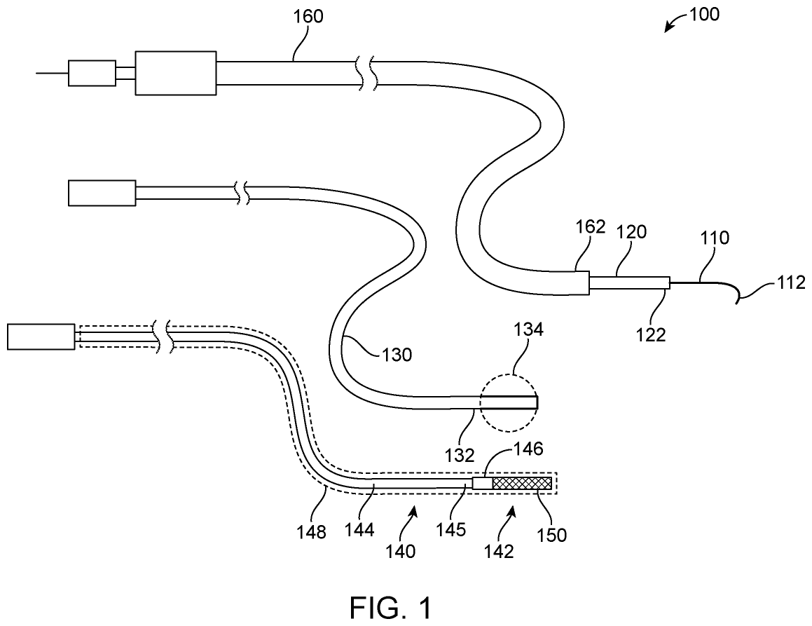

FIG. 1 illustrates a medical system 100 configured to treat an ophthalmic artery lesion.

The medical system 100 includes a guidewire 110 having a distal end 112, wherein the distal end 112 of the guidewire 110 is configured to be advanced inside a patient into an ophthalmic artery through an ostium of the ophthalmic artery until the distal end 112 of the guidewire 110 passes a lesion in the ophthalmic artery.

The medical system 100 also includes a microcatheter 120 configured to be advanced over the guidewire 110 until a distal end 122 of the microcatheter 120 is in the ophthalmic artery. The distal end 122 of the microcatheter 120 has a dimension that allows the distal end 122 of the microcatheter 120 to be placed inside the ophthalmic artery. The guidewire 110 is removable from within the microcatheter 120 while the distal end 122 of the microcatheter 120 is in the ophthalmic artery.

The medical system 100 further comprises a balloon catheter 130 configured to be advanced inside the microcatheter 120. The balloon catheter 130 comprises a balloon 134. The balloon 134 may be made from any materials, such as a polymer, a metal, or an alloy. The balloon catheter 130 has an inflation lumen, but is without any guidewire lumen (i.e., an “empty” balloon catheter 130). In some cases, the balloon catheter 130 also does not have any additional lumen that is in addition to the inflation lumen. The balloon 134 of the balloon catheter 130 is deployable outside the microcatheter 120. The balloon 134 is expandable to press against a lesion in the ophthalmic artery to create a compressed lesion.

Because the balloon catheter 130 does not have a guidewire lumen, it can be made smaller compared to another balloon catheter that has a guidewire lumen. For example, in some embodiments, the balloon catheter 130 may have an outer cross-sectional dimension (e.g., outer diameter at a proximal section or at a distal section of the balloon catheter 130) that is at least 10%, or more preferably at least 20%, or even more preferably at least 30% (e.g., 36%) smaller than the outer cross-sectional dimension of an existing balloon catheter. Also, in some embodiments, the balloon catheter 130 may have an inner cross-sectional dimension (e.g., inner diameter at a proximal section or at a distal section of the balloon catheter 130) that is at least 10%, or more preferably at least 20%, or even more preferably at least 30% (e.g., 36%) smaller than the inner cross-sectional dimension of an existing balloon catheter. However, the balloon catheter 130 may have any dimension in different embodiments. By means of non-limiting examples, the balloon catheter 130 may have a cross-sectional dimension that is anywhere from 0.5 mm to 2.0 mm. In other cases, the balloon catheter 130 may have an outer cross-sectional dimension (e.g., outer diameter) that is 0.5 mm (0.0197 inch) or smaller. Such cross-sectional dimension of the balloon catheter 130 may be the outer dimension (e.g., diameter) of a proximal section of the balloon catheter 130, such as the proximal end of the balloon catheter 130. Also, in some cases, the balloon catheter 130 may have a distal section with a cross-sectional dimension (e.g., outer diameter) that is 0.0197 inch or smaller, or more preferably 0.0178 inch or smaller, or more preferably 0.015 inch or smaller.

Also, because the balloon catheter 130 is smaller, the microcatheter 120 accommodating the balloon catheter 130 can correspondingly be made smaller as well—i.e., compared to another microcatheter configured to accommodate a balloon catheter with a guidewire lumen. For example, in some embodiments, the microcatheter 120 may have an outer cross-sectional dimension (e.g., outer diameter at a proximal section or at a distal section of the microcatheter 120) that is at least 10%, or more preferably at least 20%, or even more preferably at least 30% (e.g., 36%) smaller than the outer cross-sectional dimension of an existing microcatheter. Also, in some embodiments, the microcatheter 120 may have an inner cross-sectional dimension (e.g., inner diameter at a proximal section or at a distal section of the microcatheter 120) that is at least 10%, or more preferably at least 20%, or even more preferably at least 30% (e.g., 36%) smaller than the inner cross-sectional dimension of an existing microcatheter.

By means of non-limiting examples, the microcatheter 120 may have an internal cross-sectional dimension (e.g., diameter) that is anywhere from 0.013 inch to 0.027 inch. In other cases, the microcatheter 120 may have an internal cross-sectional dimension (e.g., diameter) that is less than 0.013 inch.

Although the balloon catheter 130 has been described as not having any guidewire lumen. In other cases, the balloon catheter 130 may include a guidewire lumen and/or a fixed wire leading tip, but these features are not required in order for the method described herein to be carried out. Also, the method described herein can still be performed if a guidewire lumen is implemented in the balloon catheter 130.

In some cases, the microcatheter 120 may have a shape (e.g., bent or curved distal segment) that facilitates entry to the ophthalmic artery. Also, in some cases, the microcatheter 120 may have an internal cross-sectional dimension (e.g., diameter) that is only slightly larger than the outer cross-sectional dimension (e.g., diameter) of the guidewire 110. This achieves a small “step-off” height between the guidewire 110 and the microcatheter 120, making access of the ophthalmic artery 12 much easier. By means of non-limiting examples, the internal cross-sectional dimension of the microcatheter 120 may be anywhere from 0.01 inch to 0.05 inch, and more preferably anywhere from 0.012 inch to 0.03 inch, and even more preferably anywhere from 0.013 inch to 0.027 inch. In some cases, the internal cross-sectional dimension of the microcatheter 120 may be a factor F times the outer cross-sectional dimension of the guidewire 110, wherein F may be any value from 1.01 to 1.10, or more preferably any value from 1.01 to 1.05. In other cases, the internal cross-sectional dimension of the microcatheter 120 may be larger or smaller than the examples described.

Optionally, the medical system 100 further includes a stent delivery device 140 configured to deliver a stent 150 in the ophthalmic artery to press against the compressed lesion. In some cases, the stent delivery device 140 includes a shaft 144 having a distal end 145, and an engagement member 146 coupled to the distal end 145 of the shaft 144. The engagement member 146 is configured to engage with the stent 150. Optionally, the stent delivery device 140 may also include a sheath 148 configured to accommodate the shaft 144, the engagement member 146, and the stent 150.

In some cases, the engagement member 146 may be configured to be inserted into a proximal end of the stent 150. When the stent 150 is in a collapsed configuration inside the microcatheter 120 and/or the sheath 148, the stent 150 is in abutment against the engagement member 146. When the stent 150 is deployed outside the microcatheter 120 and/or the sheath 148, the stent 150 extends radially away from the engagement member 146 and is detached from the engagement member 146. In other cases, the engagement member 146 may be a detachable connector that connects to the stent 150, and is capable of releasing the stent 150. Also, in further cases, the engagement member 146 may be a disintegrable link that connects the stent 150 to the shaft 144. During use, the disintegrable link is disintegrated by application of energy to thereby release the stent 150 from the shaft 144.

As shown in FIG. 1, the medical system 100 also includes a guide catheter 160 configured to accommodate the microcatheter 120, the guidewire 110, the balloon catheter 130, the stent-delivery device 140, or any or other tools. The guide catheter 160 includes a distal end 162. In some cases, the guide catheter 160 may include a marker at the distal end 162 for allowing visualization of a position of the distal end 162 in the patient via external imaging (e.g., x-ray, MRI, fluoroscope, etc.). In other cases, the guide catheter 160 is optional, and the medical system 100 may not include the guide catheter 160.

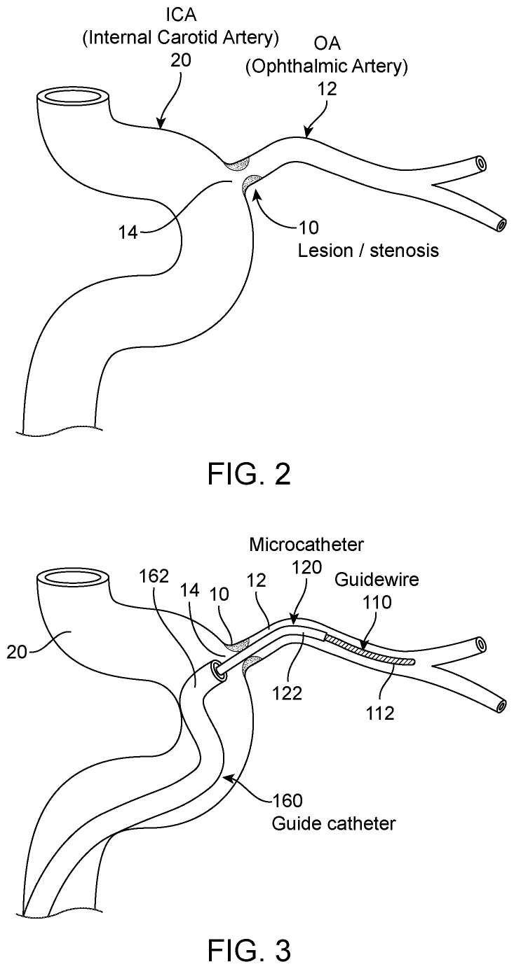

FIGS. 2-9 illustrate a method of treating an ophthalmic artery lesion. As shown in FIG. 2, the lesion 10 to be treated is located in the ophthalmic artery (OA) 12, distal to the ostium (opening) 14 of the ophthalmic artery 12. The ophthalmic artery 12 may be accessed via the internal carotid artery (ICA) 20. The ophthalmic artery 12 is relatively narrow compared to the internal carotid artery 20. Also, the ostium 14 of the ophthalmic artery 12 is quite small. Due to the mis-match between the diameters of the internal carotid artery 20 and the ophthalmic artery 12, and due to the angle formed between these two arteries, accessing the ophthalmic artery 12 has been a challenge in the past.

Referring to FIG. 3, the method of treating the ophthalmic artery lesion 10 involves advancing the distal end 112 of the guidewire 110 inside a patient into the ophthalmic artery 12 through the ostium 14 of the ophthalmic artery 12 until the distal end 112 of the guidewire 110 passes the lesion 10 in the ophthalmic artery 12. In the illustrated example, the distal end 112 of the guidewire 110 is capable of accessing the ophthalmic artery 12 from the internal carotid artery 20. For example, a distal segment of the guidewire 110 with the distal end 112 may have a certain bending angle, curvature, flexibility, etc., or any combination of the foregoing, specifically designed or selected to allow the guidewire 110 to access the ophthalmic artery 12 from the internal carotid artery 20.

Then the microcatheter 120 is advanced over the guidewire 110 until a distal end 122 of the microcatheter 120 is in the ophthalmic artery 12. The distal end 122 of the microcatheter 120 has a dimension (cross-sectional dimension) that allows the distal end 122 of the microcatheter 120 to be placed inside the ophthalmic artery 12.

In some cases, the guide catheter 160 may optionally be provided, and may be inserted into the patient before the guidewire 110 and the microcatheter 120 are advanced to the ophthalmic artery 12. The guide catheter 160 has a distal end 162, and may be advanced in the internal carotid artery 20 until the distal end 162 is outside the ostium 14 of the ophthalmic artery 12. The guidewire 110 housed in the guide catheter 160 may then be advanced to enter into the ophthalmic artery 12 through the ostium 14 of the ophthalmic artery 12, as discussed. In such cases, the act of advancing the distal end 112 of the guidewire 110 is performed while a part of the guidewire 112 is inside the guide catheter 160. The guide catheter 160 has a cross sectional dimension and/or a bending radius that renders it unable to enter into the ophthalmic artery 12. However, because the guidewire 110 is much narrower, and may have a curved distal segment designed or selected to access the ophthalmic artery 12 via the ostium 14, the guide catheter 160 can remain outside the ophthalmic artery 12 while the guide wire 110 is utilized to access the ophthalmic artery.

Also, in the situation in which the guide catheter 160 is provided, the microcatheter 120 may be advanced over the guidewire 110 while at least a part of the microcatheter 120 is inside the guide catheter 160. When the microcatheter 120 is advanced, the distal end 162 of the guide catheter 160 remains in the internal carotid artery 20 outside the ophthalmic artery 12.

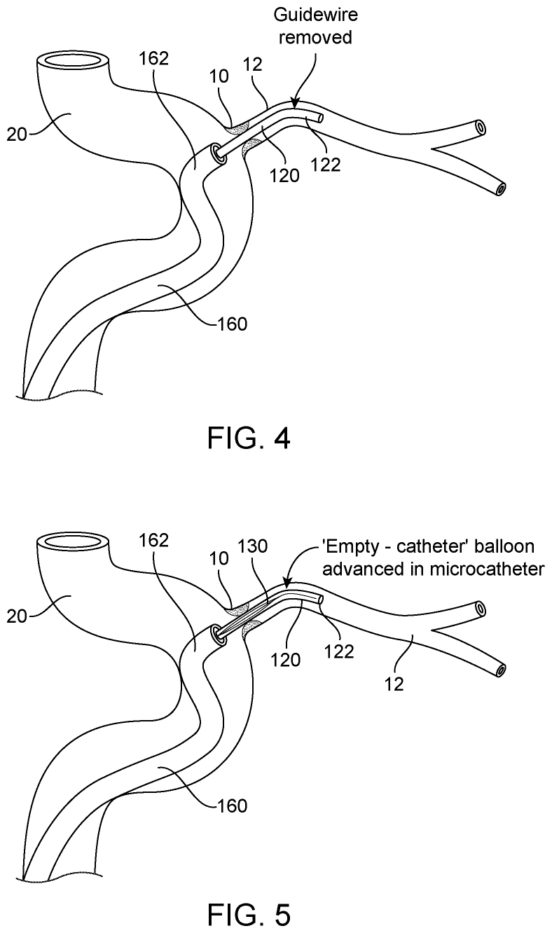

Next, referring to FIG. 4, the guidewire 110 is removed from within the microcatheter 120 while the distal end 122 of the microcatheter 120 is in the ophthalmic artery 12. The distal end 122 of the microcatheter 120 remains distal to the lesion 10 after the guidewire 110 is removed from within the microcatheter 120.

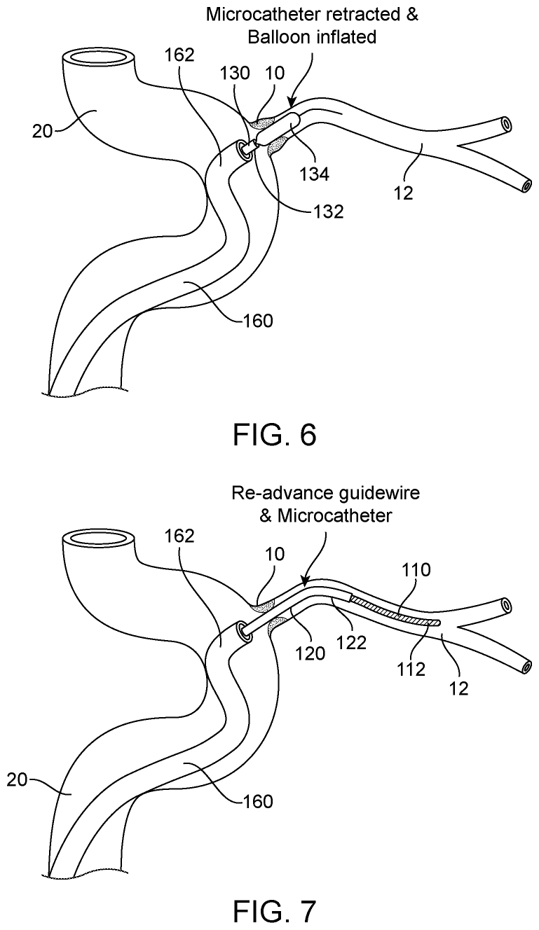

Next, referring to FIG. 5, a balloon catheter 130 is placed inside the microcatheter 120, and is advanced distally inside the microcatheter 120. The same lumen of the microcatheter 120 that was utilized for housing the guidewire 110 may be utilized again-this time for the balloon catheter 130. This is possible due to the removal of the guidewire 110. In the illustrated example, the balloon catheter 130 comprises a distal end 132 (shown in FIG. 6), and a balloon 134 at the distal end 132 of the balloon catheter 130. The balloon catheter 130 is without any guidewire lumen. In other cases, the balloon catheter 130 may include one or more lumens, such as a guidewire lumen.

Next, as shown in FIG. 6, the balloon 134 of the balloon catheter 130 is deployed outside the microcatheter 120. In some cases, such may be accomplished by retracting the microcatheter 120 proximally relative to the balloon catheter 130, until the balloon 134 exits from the distal end 122 of the microcatheter 120. In other cases, such may be accomplished by advancing the balloon catheter 130 distally relative to the microcatheter 120, until the balloon 134 exits from the distal end 122 of the microcatheter 120. In further cases, the microcatheter 120 may be retracted proximally relative to the balloon catheter 130, and the balloon catheter 130 may be simultaneously advanced distally relative to the microcatheter 120, until the balloon 134 exits from the distal end 122 of the microcatheter 120. As shown in the figure, the deployed position for the balloon 134 is at the lesion site (at the location of the lesion). Alternatively, the balloon 134 may be deployed at a position that is distal to the lesion site. In such cases, after the balloon 134 is deployed, the balloon catheter 130 may be retracted proximally to align the balloon 134 with the lesion 10 to be treated.

After the balloon is desirably positioned, the balloon 134 is expanded to press against the lesion 10 in the ophthalmic artery 12 to create a compressed lesion.

It should be noted that the above technique of placing the balloon catheter 130 in the ophthalmic artery 12 is advantageous. By maintaining the distal end 122 of the microcatheter 120 inside the ophthalmic artery 12, and by removing the guidewire 110 from the lumen of the microcatheter 120, the same lumen that was used for the guidewire 110 becomes available for housing and guiding the balloon catheter 130 to the target site inside ophthalmic artery 12. Accordingly, the act of advancing the balloon catheter 130 inside the microcatheter 120 may be performed without placing the balloon catheter 130 over the guidewire 110 or any other guidewire. No guidewire is needed for navigating the balloon catheter 130 to the ophthalmic artery 12 because the distal end 122 of the microcatheter 120 with an available lumen is already inside the ophthalmic artery 12, and so the balloon catheter 130 can simply be delivered to the target site until the balloon 134 is inside the ophthalmic artery 12.

In some cases, to assist with the deployment and/or placement of the balloon 134, the balloon 134 and/or the distal end 132 of the balloon catheter 130 may include one or more markers for visualization under imaging guidance. The marker(s) may be radiopaque markers, for example. The marker(s) may be of the type that allow visualization under MRI, x-ray, fluoroscope, etc., or any of other types of imaging.

After the lesion 10 is compressed, the balloon 134 is deflated, and the balloon catheter 130 is removed from within the patient. The deflation of the balloon 134 may be accomplished by removing the inflation fluid inside the balloon 134, such as by suction. The removal of the balloon catheter 130 may be achieved by retracting the balloon catheter 130 proximally until the balloon catheter 130 exits the patient. In some cases, if the guide catheter 160 is inside the internal carotid artery 20 and is containing a part of the balloon catheter 130, the balloon catheter 130 may be retracted proximally relative to the guide catheter 160 to remove the balloon catheter 130 from within the patient. The guide catheter 160 may then be retracted proximally to remove the guide catheter 160 from within the patient.

In some cases, the compression of the ophthalmic artery lesion 10 may be considered as a treatment of the ophthalmic artery lesion 10. In other cases, the treatment of the ophthalmic artery lesion 10 may involve delivery of a stent to maintain the lumen of the ophthalmic artery at the lesion site.

FIG. 7 shows that the method of treating the ophthalmic artery lesion 10 may optionally further include advancing the distal end 112 of the guidewire 110 inside the patient again into the ophthalmic artery 12 through the ostium 14 of the ophthalmic artery 12 until the distal end 112 of the guidewire 110 passes the compressed lesion 10.

Then the microcatheter 120 is advanced over the guidewire 110 again until the distal end 122 of the microcatheter 120 is in the ophthalmic artery 12. In some cases, the microcatheter 120 may be advanced until the distal end 122 of the microcatheter 120 is distal to the ophthalmic artery lesion 10.

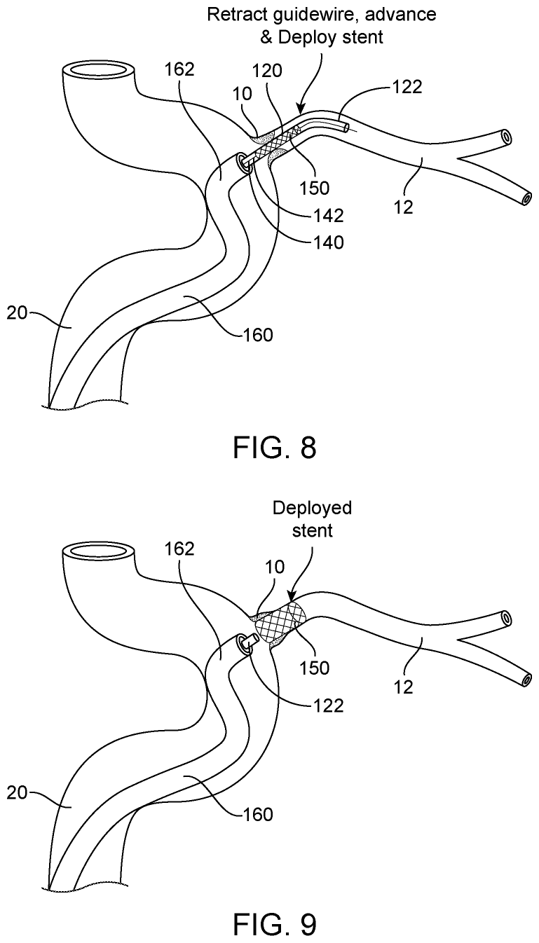

Referring to FIG. 8, next, a stent-delivery device 140 with a stent 150 is placed inside the microcatheter 120, and is advanced inside the microcatheter 120 until the distal end 142 of the stent-delivery device 140 reaches the target site inside the ophthalmic artery 12. In some cases, the stent-delivery device 140 comprises the shaft 144 with the engagement member 146 attached to the distal end 142 of the shaft 144. The engagement member 146 is configured to engage the stent 150. For example, the engagement member 146 may be inserted into a lumen of the stent 150 at a proximal end of the stent 150. As another example, the engagement member 146 may have a connector that releasably connects to the stent 150. As a further example, the engagement member 146 may have a distal end that abuts the proximal end of the stent 150. Optionally, the stent-delivery device 140 may further include the 148 sheath for housing the shaft, the engagement member, and the stent 150.

As shown in FIG. 9, the stent-delivery device 140 is used to deploy the stent 150 out of the distal end 122 of the microcatheter 120. As shown in the figure, the deployed stent 150 compresses against the lesion 10 in the ophthalmic artery 12. Also, the deployed stent 150 is completely within the ophthalmic artery 12 such that no part of the deployed stent 150 protrudes into the internal carotid artery 20.

In some cases, the deployment of the stent 150 may be accomplished by advancing the shaft of the stent-delivery device 140 distally relative to the microcatheter 120. The advancement of the shaft of the stent-delivery device 140 causes the engagement member of the stent-delivery device 140 to push the stent 150 distally. The advancement of the shaft of the stent-delivery device 140 distally is performed until the entirety of the stent 150 is outside the microcatheter 120. When the stent 150 is contained in the lumen of the microcatheter 120, the stent 150 has a collapsed configuration (like that shown in FIG. 8). After the stent 150 exits out of the microcatheter 120, the stent 150 assumes an expanded configuration (like that shown in FIG. 9). The expanded stent 150 provides a radial force to push the lesion 10 radially towards the wall of the ophthalmic artery 10.

In other cases, if the stent-delivery device 140 includes the sheath 148 for housing the shaft 144, the engagement member 146, and the stent 150, then the placement of the distal end 142 of the stent-delivery device 140 at the lesion site involves placing the distal end of the sheath 148 at the lesion site. In such cases, after the distal end of the sheath 148 of the stent-delivery device 140 is positioned at a desired position relative to the ophthalmic artery 12, the microcatheter 120 may be retracted proximally to cause the distal end of the sheath 148 to exit from the distal end 122 of the microcatheter 120. The retraction of the microcatheter 120 is performed while the distal end of the sheath 148 of the stent-delivery device 140 remains inside the ophthalmic artery 12. Alternatively, the sheath 148 of the stent-delivery device 140 may be advanced distally relative to the microcatheter 120 to exit out of the distal end 122 of the microcatheter 120, and is placed at a desired position relative to the ophthalmic artery 12. The desired position of the distal end of the sheath 148 is considered achieved when the stent 150 contained inside the sheath 148 is in/across the lesion 10 while contained inside the sheath 148. After the distal end of the sheath 148 has been desirably placed in the ophthalmic artery 12, the distal end of the sheath 148 is then retraced proximally relative to the stent 150 to deploy the stent 150. In particular, the sheath 148 of the stent-delivery device 140 may be retracted proximally while maintaining the shaft 144 of the stent-delivery device 140 at a desired position. The retraction of the sheath 148 of the stent-delivery device 140 is performed until the distal end of the sheath 148 is proximal to the stent 150, thereby deploying the stent 150 out of the lumen of the sheath 148. The sheath 148 may be further retracted to exit from the lumen of the ophthalmic artery 12.

In some cases, to assist with the placement of the distal end of the sheath 148 of the stent-delivery device 140 and/or with the deployment of the stent 150, the distal end of the sheath 148 of the stent-delivery device 140 and/or the stent 150 may include one or more markers for visualization under imaging guidance. The marker(s) may be radiopaque markers, for example. The marker(s) may be of the type that allow visualization under MRI, x-ray, fluoroscope, etc., or any of other types of imaging.

Also, in other cases, instead of using the microcatheter 120 to transport the stent-delivery device 140 and the stent 150, a separate catheter, such as a stent catheter may be used instead. In such cases, after the lesion 10 has been compressed by the balloon shown in FIG. 6, and after the balloon catheter 130 has been removed from the microcatheter 120, the microcatheter 120 may then be removed from the patient. Then the guidewire 110 may be advanced into the ophthalmic artery 12, and the stent catheter (instead of the microcatheter 120) may be advanced over the guidewire 110 until the distal end of the stent catheter is in the ophthalmic artery 12 (like that shown in FIG. 7, except the microcatheter 120 is replaced with the stent catheter). In some cases, after the distal end of the stent catheter is placed in the ophthalmic artery 12, the guidewire 110 may be removed from within the stent catheter (e.g., by retracting the guidewire 110 proximally) while the distal end of the stent catheter is in the ophthalmic artery 12. Then the shaft 144 of the stent-delivery device 140 and the stent 150 may be inserted into the stent catheter, and are then advanced distally, unit the stent 150 is deployed out of the distal end of the stent catheter. In other cases, the stent catheter may include a lumen dedicated for housing the guidewire 110. In such cases, the guidewire 110 may not need to be removed from the stent catheter before the stent 150 is delivered out of the distal end of the stent catheter, and the distal end 112 of the guidewire 110 remains inside the ophthalmic artery 12 while the stent 150 is being delivered inside the ophthalmic artery 12. In further cases, the stent catheter may include a lumen for housing both the guidewire 110 and the stent 150. In such cases, the guidewire 110 also may not need to be removed from the stent catheter before the stent 150 is delivered out of the distal end of the stent catheter, and the distal end 112 of the guidewire 110 remains inside the ophthalmic artery 12 while the stent 150 is being delivered inside the ophthalmic artery 12. Furthermore, in other cases, the stent catheter may already come with the shaft 144, the engagement member 146, and the stent 150. In such cases, the shaft 144 and the stent 150 do not need to be inserted into the stent catheter.

The above method for treating the ophthalmic artery lesion 10 is performed using the medical system 100 that includes the guidewire 110, the microcatheter 120, and the balloon catheter 130. In the cases in which the method also includes delivery of the stent 150 to treat the ophthalmic artery lesion 10, the medical system 100 may optionally also include the stent delivery device 140 configured to deliver the stent 150 into the ophthalmic artery 12. It should be noted that the system 100 for performing the method described herein should not be limited to having the configurations or features described. For example, in other cases, the guide catheter 160 of the medical system 100 for performed the method described herein may be steerable. In such cases, the guide catheter 160 may be inserted into the patient and its distal end 162 is steered as the guide catheter 160 is being advanced distally to reach a target site (the internal carotid artery 20.

The above technique of treating the ophthalmic artery lesion 10 is advantageous. The balloon catheter 130 without a guidewire lumen (the “empty” balloon catheter) can be made smaller (compared to another balloon catheter that has a guidewire lumen or an additional lumen that is in addition to the inflation lumen), thereby making it easier to be navigated from the internal carotid artery 20 into the ophthalmic artery 12 via the ostium 14. Also, because the balloon catheter 130 is smaller, the microcatheter 120 containing the balloon catheter 130 can also be made smaller (compared to another microcatheter that is configured to carry a balloon catheter with guidewire lumen or a balloon catheter with an additional lumen that is in addition to the inflation lumen). Thus, the distal end 122 of the microcatheter 120 and the distal end 132 of the balloon catheter 130 can both access into the ophthalmic artery 12 and easily cross the ophthalmic artery lesion 10 due to their smaller sizes. This is also advantageous compared to another technique in which the microcatheter does not enter the ophthalmic artery, and remains outside the ostium of the ophthalmic artery. By having he distal end 122 of the microcatheter 120 cross the ophthalmic artery lesion 10, the microcatheter 120 itself can be used to delivery different tools, e.g., balloon catheter 130, stent-delivery device 140, stent 150, etc. to treat the ophthalmic artery lesion 10. This is advantageous because the microcatheter 120 is relatively more navigable than the balloon catheter 130, and has a smaller crossing profile. Furthermore, because the distal end 122 of the microcatheter 120 can be delivered past the ophthalmic artery lesion 10, the stent 150 can be delivered completely inside the ophthalmic artery 12 without any portion of the stent 150 protruding into the internal carotid artery 20.

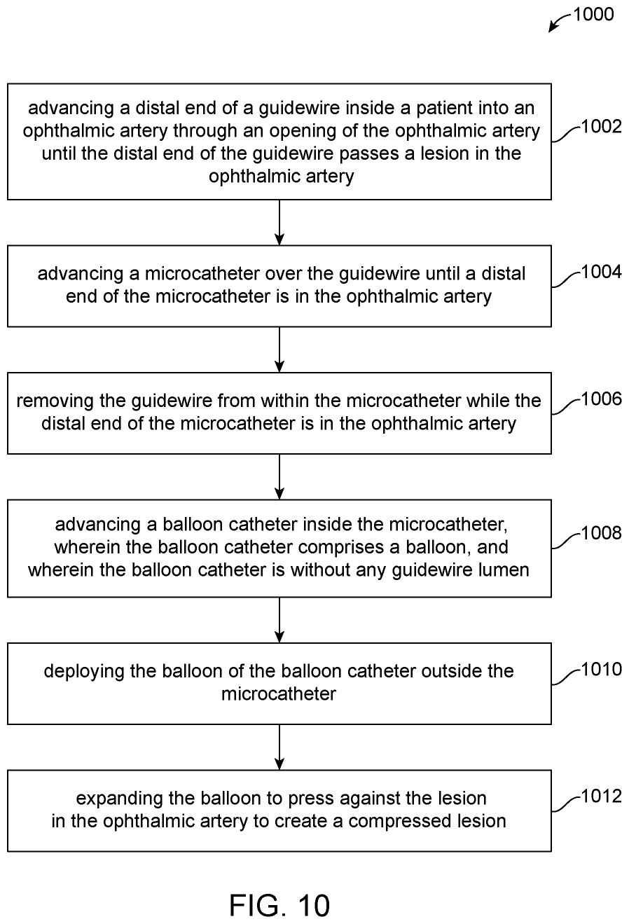

FIG. 10 illustrates a method 1000 of treating an ophthalmic artery lesion. The method 1000 includes: advancing a distal end of a guidewire inside a patient into an ophthalmic artery through an ostium of the ophthalmic artery until the distal end of the guidewire passes a lesion in the ophthalmic artery (item 1002); advancing a microcatheter over the guidewire until a distal end of the microcatheter is in the ophthalmic artery (item 1004); removing the guidewire from within the microcatheter while the distal end of the microcatheter is in the ophthalmic artery (item 1006); advancing a balloon catheter inside the microcatheter, wherein the balloon catheter comprises a balloon, and wherein the balloon catheter is without any guidewire lumen (item 1008); deploying the balloon of the balloon catheter outside the microcatheter (item 1010); and expanding the balloon to press against the lesion in the ophthalmic artery to create a compressed lesion (item 1012).

Optionally, the method 1000 further includes: deflating the balloon; and removing the balloon catheter from within the patient.

Optionally, the method 1000 further includes advancing the distal end of the guidewire inside the patient again into the ophthalmic artery through the ostium of the ophthalmic artery until the distal end of the guidewire passes the compressed lesion.

Optionally, the method 1000 further includes advancing the microcatheter over the guidewire again until the distal end of the microcatheter is in the ophthalmic artery.

Optionally, the method 1000 further includes: placing a stent-delivery device inside the microcatheter; and using the stent-delivery device to deploy a stent out of the distal end of the microcatheter, wherein the deployed stent compresses against the lesion.

Optionally, the method 1000 further includes advancing a stent catheter over the guidewire until a distal end of the stent catheter is in the ophthalmic artery.

Optionally, the method 1000 further includes: removing the guidewire from within the stent catheter while the distal end of the stent catheter is in the ophthalmic artery; and deploying a stent out of the distal end of the stent catheter, wherein the deployed stent compresses against the lesion.

Optionally, the method 1000 further includes deploying a stent out of the distal end of the stent catheter, wherein the deployed stent compresses against the lesion.

Optionally, the method 1000 further includes advancing a guide catheter inside the patient until a distal end of the guide catheter is in an internal carotid artery facing the ostium of the ophthalmic artery;

Optionally, in the method 1000, the act of advancing the distal end of the guidewire is performed while a part of the guidewire is inside the guide catheter.

Optionally, in the method 1000, the act of advancing the microcatheter over the guidewire is performed while at least a part of the microcatheter is inside the guide catheter.

Optionally, in the method 1000, when the microcatheter is advanced, the distal end of the guide catheter remains in the internal carotid artery outside the ophthalmic artery.

Optionally, in the method 1000, the act of advancing the balloon catheter inside the microcatheter is performed without placing the balloon catheter over the guidewire or any other guidewire.

Optionally, in the method 1000, the act of deploying the balloon comprises retracting the microcatheter proximally or advancing the balloon catheter distally.

Optionally, the method 1000 is performed using a medical system that comprises the guidewire, the microcatheter, and the balloon catheter.

Optionally, in the method 1000, the distal end of the microcatheter has a dimension that allows the distal end of the microcatheter to be placed inside the ophthalmic artery.

Optionally, in the method 1000, the distal end of the guidewire is capable of accessing the ophthalmic artery from the internal carotid artery.

Optionally, in the method 1000, the medical system also comprises a stent delivery device configured to deliver a stent into the ophthalmic artery.

It should be noted that the method described herein for treating ophthalmic artery lesion may be similarly applied to treat other medical conditions (e.g., atherosclerotic conditions) in different parts of patients.

As used in this specification, the terms “apparatus”, “device”, “system” may refer to one or more product(s), one or more component(s), a part of a product, or a part of a component. In some cases, any of these terms may refer to two or more components that are coupled to each other, and/or that have functional and/or positional relationship with respect to each other. Also, in some cases, these terms are synonymous to each other.

Furthermore, as used in this specification, the term “distal end” of an item (e.g., guidewire, microcatheter, balloon catheter, stent, guide catheter, etc.) may refer to any part of such item that is within ⅓, or ¼, or ⅕, or ⅙, or 1/7, or ⅛, or 1/9, or 1/10 of a total length of the item measured from a distal tip of the item. Similarly, the term “proximal end” of an item (e.g., guidewire, microcatheter, balloon catheter, stent, guide catheter, etc.) may refer to any part of such item that is within ⅓, or ¼, or ⅕, or ⅙, or 1/7, or ⅛, or 1/9, or 1/10 of a total length of the item measured from a proximal tip of the item.

Although particular embodiments have been shown and described, it will be understood that it is not intended to limit the claimed inventions to the preferred embodiments, and it will be obvious to those skilled in the art that various changes and modifications may be made without department from the scope of the claimed inventions. The specification and drawings are, accordingly, to be regarded in an illustrative rather than restrictive sense. The claimed inventions are intended to cover alternatives, modifications, and equivalents.

1. A method of treating an ophthalmic artery lesion, comprising:
advancing a distal end of a guidewire inside a patient into an ophthalmic artery through an ostium of the ophthalmic artery until the distal end of the guidewire passes a lesion in the ophthalmic artery;
advancing a microcatheter over the guidewire until a distal end of the microcatheter is in the ophthalmic artery;
removing the guidewire from within the microcatheter while the distal end of the microcatheter is in the ophthalmic artery;
advancing a balloon catheter inside the microcatheter, wherein the balloon catheter comprises a balloon, and wherein the balloon catheter is without any lumen for accommodating the guidewire or any guiding structure;
deploying the balloon of the balloon catheter outside the microcatheter; and expanding the balloon to press against the lesion in the ophthalmic artery to create a compressed lesion.

2. The method of claim 1, further comprising:
deflating the balloon; and
removing the balloon catheter from within the patient.

3. The method of claim 1, further comprising advancing the distal end of the guidewire inside the patient again into the ophthalmic artery through the ostium of the ophthalmic artery until the distal end of the guidewire passes the compressed lesion.

4. The method of claim 3, further comprising advancing the microcatheter over the guidewire again until the distal end of the microcatheter is in the ophthalmic artery.

5. The method of claim 4, further comprising:
placing a stent-delivery device inside the microcatheter; and
using the stent-delivery device to deploy a stent out of the distal end of the microcatheter, wherein the deployed stent compresses against the lesion.

6. The method of claim 3, further comprising advancing a stent catheter over the guidewire until a distal end of the stent catheter is in the ophthalmic artery.

7. The method of claim 6, further comprising:
removing the guidewire from within the stent catheter while the distal end of the stent catheter is in the ophthalmic artery; and
deploying a stent out of the distal end of the stent catheter, wherein the deployed stent compresses against the lesion.

8. The method of claim 6, further comprising deploying a stent out of the distal end of the stent catheter, wherein the deployed stent compresses against the lesion.

9. The method of claim 1, further comprising advancing a guide catheter inside the patient until a distal end of the guide catheter is in an internal carotid artery facing the ostium of the ophthalmic artery.

10. The method of claim 9, wherein the act of advancing the distal end of the guidewire is performed while a part of the guidewire is inside the guide catheter.

11. The method of claim 9, wherein the act of advancing the microcatheter over the guidewire is performed while at least a part of the microcatheter is inside the guide catheter.

12. The method of claim 11, wherein when the microcatheter is advanced, the distal end of the guide catheter remains in the internal carotid artery outside the ophthalmic artery.

13. The method of claim 1, wherein the act of advancing the balloon catheter inside the microcatheter is performed without placing the balloon catheter over the guidewire.

14. The method of claim 1, wherein the act of deploying the balloon comprises retracting the microcatheter proximally or advancing the balloon catheter distally.

15. The method of claim 1, wherein the method is performed using a medical system that comprises the guidewire, the microcatheter, and the balloon catheter.

16. The method of claim 15, wherein the distal end of the microcatheter has a dimension that allows the distal end of the microcatheter to be placed inside the ophthalmic artery.

17. The method of claim 15, wherein the distal end of the guidewire is capable of accessing the ophthalmic artery from the internal carotid artery.

18. The method of claim 15, wherein the medical system also comprises a stent delivery device configured to deliver a stent into the ophthalmic artery.

19. A medical system for treating an ophthalmic artery lesion, the medical system comprising:
a guidewire having a distal end, wherein the distal end of the guidewire is configured to be advanced inside a patient into an ophthalmic artery through an ostium of the ophthalmic artery until the distal end of the guidewire passes a lesion in the ophthalmic artery; and
a microcatheter configured to be advanced over the guidewire until a distal end of the microcatheter is in the ophthalmic artery;
wherein the guidewire is removable from within the microcatheter while the distal end of the microcatheter is in the ophthalmic artery;
wherein the medical system further comprises a balloon catheter configured to be advanced inside the microcatheter, wherein the balloon catheter comprises a balloon, wherein the balloon catheter is without any lumen for accommodating the guidewire or any guiding structure;
wherein the balloon of the balloon catheter is deployable outside the microcatheter; and
wherein the balloon is expandable to press against the lesion to create a compressed lesion.

20. The medical system of claim 19, further comprising a stent delivery device configured to deliver a stent in the ophthalmic artery to press against the compressed lesion.
